(19) United States
(12) Patent Application Publication
(10) Pub. No.: US 2020/0137971 A1
(43) Pub. Date: May 7, 2020

(54) REGENERATION OF CEREALS

(71) Applicant: BASF SE, Ludwigshafen (DE)

(72) Inventors: Els Roggeman, Gent (BE); Michiel Vander Auwermeulen, Gent (BE)

(21) Appl. No.: 16/625,048

(22) PCT Filed: Apr. 18, 2018
(86) PCT No.: PCT/EP2018/059845

§ 371 Date: Dec. 20, 2019

(30) Foreign Application Priority Data
Jun. 26, 2017 (EP) 17177834.3

(51) Int. Cl. A01H 4/00; A01H 5/00; A01H 6/46

(52) U.S. Cl. CPC A01H 4/008; A01H 6/4678; A01H 5/00; A01H 4/005

(57) ABSTRACT

The present invention relates to materials and methods for temporary storage of a cereal plant being regenerated, for the production and regeneration of cereal plants, for increasing yield of a regenerated cereal plant, such as wheat plants. In particular, a step of temporarily immersing plantlets in liquid medium at low temperature is provided.

### FIELD OF THE INVENTION

The present invention relates to materials and methods for temporary storage of a cereal plant during the process of regeneration, for the production and regeneration of cereal plants, for increasing yield of a regenerated cereal plant, such as wheat plants. In particular, a step of temporarily immersing plantlets in liquid medium at low temperature is provided.

### BACKGROUND

Plant regeneration is a tissue culture method which comprises regenerating a plant from a tissue made of dedifferentiated cells, the callus, from a somatic embryo or from an immature embryo. This de novo organogenesis occurs thanks to the successive application of growth media supplemented with hormones so as to first initiate leaves and then initiate the development of roots, resulting in an in vitro plantlet. The obtained plantlet can then be transferred to soil to further grow and produce seeds.

In the context of experiments aiming at altering the genetic composition of the plant, like somatic hybridization (production of a hybrid by protoplasts fusion), mutagenesis, targeted genome engineering or production of transgenic plants, a selection can be performed on the regenerating calli by adding a selective pressure to their environment. For example, NaCl can be added to the media to selectively regenerate plantlets exhibiting salt tolerance (e.g. Zair et al. 2003 Plant Cell Tissue Organ Cult 73:237-244) or a herbicide can be applied to either selectively regenerate plantlets which exhibit a resistance to the applied herbicide (WO 2013/127766). However, when the expected genetic alteration does not result in a trait that can be assessed by simply modifying the environment of the callus or plantlets, or when more extensive information is required, like copy number or integration site(s) in the genome, destructive molecular analyses are required. The in vitro plantlet obtained by regeneration corresponds to the earliest stage when samples can be collected to perform such analyses. However, the time required to complete and analyze the results of said molecular analyses is such that the selection of the relevant plants can only occur after the plantlets have been transferred to soil, causing additional workload and practical issues such as space availability in a greenhouse. An alternative would be to temporarily store the plantlets, in a way allowing mass storage, prior to the transfer to the greenhouse, until the outcome of the analyses performed is available. Although methods related to cryopreservation or maintenance by subculture of calli have been long available (e.g. Green and Phillips, 1975; Chen et al. 1985) they are inappropriate for plantlets.

There remains thus a need to develop a method allowing temporary storage of plantlets during the in vitro regeneration process, prior to the transfer to the greenhouse. This and other problems are solved as hereinafter described, particularly in the different embodiments, examples and claims.

### SUMMARY

In one aspect, the invention provides a method of temporarily storing a cereal plant being regenerated through tissue culture comprising the steps of (a) regenerating a plantlet from a callus, a somatic embryo or an immature embryo, and (b) temporarily immersing the plantlet in liquid medium at low temperature. In another embodiment the cereal plant is a wheat plant. Further embodiments provide that said plantlet is immersed for a period between 4 and 10 weeks and that the liquid medium comprises a carbon source, such as maltose. In another embodiment, said temperature is between 4° C. and 15° C., preferably 10° C.

In another aspect, the invention provides a method of regenerating a cereal plant through tissue culture comprising the steps of (a) regenerating a plantlet from a callus, a somatic embryo or an immature embryo, and (b) temporarily immersing the plantlet in liquid medium at low temperature. In another embodiment the cereal plant is a wheat plant. Further embodiments provide that said plantlet is immersed for a period between 4 and 10 weeks and that the liquid medium comprises a carbon source, such as maltose. In another embodiment, said temperature is between 4° C. and 15° C., preferably 10° C.

Further provided is a method of increasing yield of a cereal plant regenerated through tissue culture comprising the steps of (a) regenerating a plantlet from a callus, a somatic embryo or an immature embryo, and (b) temporarily immersing the plantlet in liquid medium at low temperature. In another embodiment the cereal plant is a wheat plant. Further embodiments provide that said plantlet is immersed for a period between 4 and 10 weeks and that the liquid medium comprises a carbon source, such as maltose. In another embodiment, said temperature is between 4° C. and 15° C., preferably 10° C. In yet another embodiment, the increased yield is seed yield, with preferentially a yield increase of at least 2.1 fold compared to plants regenerated through tissue culture that were not temporarily immersed in liquid medium but temporarily stored at low temperature. In another embodiment, the increased yield is due to an increase of the thousand seed weight, with preferentially a yield increase of at least 5% compared to plants regenerated through tissue culture that were not temporarily immersed in liquid medium but were temporarily stored at low temperature.

Yet another embodiment provides a method of producing a cereal plant through tissue culture comprising the steps of (a) regenerating a plantlet from a callus, a somatic embryo or an immature embryo, and (b) temporarily immersing the plantlet in liquid medium at low temperature. In another embodiment the cereal plant is a wheat plant. Further embodiments provide that said plantlet is immersed for a period between 4 and 10 weeks and that the liquid medium comprises a carbon source, such as maltose. In another embodiment, said temperature is between 4° C. and 15° C., preferably 10° C.

Also provided is a cereal plant obtained by the methods described above. In a further embodiment, said cereal plant is a wheat plant. In yet another embodiment, said plant has an increased yield compared to a plant regenerated through tissue culture that were not temporarily immersed in liquid medium but were temporarily stored at low temperature. In one embodiment the increased yield is seed yield, with preferentially a yield increase of at least 2.1 fold compared to plants regenerated through tissue culture that were not temporarily immersed in liquid medium but were temporarily stored at low temperature. In another embodiment, the increased yield is due to an increase of the thousand seed weight, with preferentially a yield increase of at least 5% compared to plants regenerated through tissue culture that were not temporarily immersed in liquid medium but were temporarily stored at low temperature.

### DETAILED DESCRIPTION

This invention is based on the serendipitous observation that cereal plantlets, such as wheat plantlets, that were being regenerated through tissue culture could be temporarily stored by immersing them in liquid medium at low temperature. It was even more surprising to find that the plants regenerated through tissue culture that were temporarily immersed in liquid medium at low temperature had a higher yield than those that were not temporarily immersed in liquid medium but were temporarily stored at low temperature.

In one aspect, the invention provides a method of temporarily storing a cereal plant being regenerated through tissue culture comprising the steps of (a) regenerating a plantlet from a callus, an somatic embryo or an immature embryo, and (b) temporarily immersing the plantlet in liquid medium at low temperature. In one embodiment the cereal plant is a wheat plant. Further embodiments provide that said plantlet is immersed for a period between 4 and 10 weeks and that the liquid medium comprises a carbon source, such as maltose. In another embodiment, said temperature is between 4° C. and 15° C., preferably 10° C.

The plant regeneration process consists of growing a plant or plantlet from a plant cell or a plant tissue. Regeneration through tissue culture indicates that this regeneration process occurs in vitro, prior to the transfer to the greenhouse. The plant tissue can be a callus, a somatic embryo or an immature embryo. The term callus means an undifferentiated mass of plant cells or plant tissues, the term somatic embryo means an embryo derived from a somatic cell, i.e. from a cell which is not normally involved in the development of embryos, while the term immature embryo refers an embryo isolated from an immature seed. Methods for excision of plant immature embryos are well known in the art (for example Yuji Ishida et al. 2015, Methods in Molecular Biology, 1223: 189-198).

“Storing” as used herein means reducing significantly the growth of the plantlet without negatively affecting its viability. Preserving, maintaining, setting aside and conserving are equivalent terms. During storage the plant growth is reduced by at least about 80%, at least about 90%, at least about 95%, at least about 99% at least about 100%. Plant growth may be abolished during the storage.

“Temporarily” as used herein refers to the duration of the storage being about 4 weeks, about 5 weeks, about 6 weeks, about 7 weeks, about 8 weeks, about 9 weeks, about 10 weeks, about 11 weeks, about 12 weeks. The duration of the storage may also last between about 4 and about 12 weeks, between about 5 and about 12 weeks, between about 6 and about 12 weeks, between about 7 and about 12 weeks, between about 8 and about 12 weeks, between about 8 and about 12 weeks, between about 9 and about 12 weeks, between about 10 and about 12 weeks, between about 11 and about 12 weeks, between about 4 and about 11 weeks, between about 5 and about 11 weeks, between about 6 and about 11 weeks, between about 7 and about 11 weeks, between about 8 and about 11 weeks, between about 8 and about 11 weeks, between about 9 and about 11 weeks, between about 10 and about 11 weeks, between about 4 and about 10 weeks, between about 5 and about 10 weeks, between about 6 and about 10 weeks, between about 7 and about 10 weeks, between about 8 and about 10 weeks, between about 8 and about 10 weeks, between about 9 and about 10 weeks, between about 4 and about 9 weeks, between about 5 and about 9 weeks, between about 6 and about 9 weeks, between about 7 and about 9 weeks, between about 8 and about 9 weeks, between about 4 and about 8 weeks, between about 5 and about 8 weeks, between about 6 and about 8 weeks, between about 7 and about 8 weeks, between about 4 and about 7 weeks, between about 5 and about 7 weeks, between about 6 and about 7 weeks, between about 4 and about 6 weeks, between about 5 and about 6 weeks, between about 4 and about 5 weeks.

“Immersing” as used herein means adding liquid medium onto the plantlet so as to cover at least about 70% of its shoot and leaves, at least about 80% of its shoot and leaves, at least about 90% of its shoot and leaves, at least about 95% of its shoot and leaves, at least about 98% of its shoot and leaves, at least about 99% of its shoot and leaves, at least about 100% of its shoot and leaves. Immerging, dipping, plunging, sinking are equivalent terms. The plantlet may also be submersed in the liquid medium, i.e. be fully immersed in liquid medium.

“Liquid medium” as used herein is a medium to which no thickening or solidifying agent, as for example agar, has been added. Liquid growth medium, liquid culture medium are equivalent terms. The liquid medium comprises at least water. The liquid medium may also comprise a carbon source. Vitamins, salts, metals and phytohormones may be added to said liquid medium.

“Carbon source” as used herein refer to carbohydrates which can be used in tissue culture. The carbon source may be a sugar: a monosaccharose hexose—like glucose, fructose, galactose, and mannose—, a pentose—like arabinose, ribose, xylose—, a disaccharide—like sucrose, maltose, lactose, cellobiose, trehalose—, a trisaccharide—like raffinose—. The carbon source may also be sugar alcohol, like sorbitol, glycerol and mannitol. The carbon source is provided in the liquid medium at a concentration ranging between about 1 and about 5% weight, preferentially between about 2% and about 4%.

“Low temperature” as used herein mean cool, non-freezing temperature. Said low temperature can be about 4° C., about 5° C., about 6° C., about 7° C., about 8° C., about 9° C., about 10° C., about 11° C., about 12° C., about 13° C., about 14° C., about 15° C. Preferentially the low temperature is about 10° C. The low temperature may also be between about 4° C. and about 15° C., between about 5° C. and about 15° C., between about 6° C. and about 15° C., between about 7° C. and about 15° C., between about 8° C. and about 15° C., between about 9° C. and about 15° C., between about 10° C. and about 15° C., between about 11° C. and about 15° C., between about 12° C. and about 15° C., between about 13° C. and about 15° C., between about 14° C. and about 15° C., between about 4° C. and about 14° C., between about 5° C. and about 14° C., between about 6° C. and about 14° C., between about 7° C. and about 14° C., between about 8° C. and about 14° C., between about 9° C. and about 14° C., between about 10° C. and about 14° C., between about 11° C. and about 14° C., between about 12° C. and about 14° C., between about 13° C. and about 14° C., between about 4° C. and about 13° C., between about 5° C. and about 13° C., between about 6° C. and about 13° C., between about 7° C. and about 13° C., between about 8° C. and about 13° C., between about 9° C. and about 13° C., between about 10° C. and about 13° C., between about 11° C. and about 13° C., between about 12° C. and about 13° C., between about 4° C. and about 11° C., between about 5° C. and about 11° C., between about 6° C. and about 11° C., between about 7° C. and about 11° C., between about 8° C. and about 11° C., between about 9° C. and about 11° C., between about 10° C. and about 11° C., between about 4° C. and about 10° C., between about 5° C. and about 10° C., between about 6° C. and about 10° C., between about 7° C. and about 10° C., between about 8° C. and about 10° C., between about 9° C. and about 10° C., between about 4° C. and about 9° C., between about 5° C. and about 9° C., between about 6° C. and about 9° C., between about 7° C. and about 9° C., between about 8° C. and about 9° C., between about 4° C. and about 8° C., between about 5° C. and about 8° C., between about 6° C. and about 8° C., between about 7° C. and about 8° C., between about 4° C. and about 7° C., between about 5° C. and about 7° C., between about 6° C. and about 7° C., between about 4° C. and about 6° C., between about 5° C. and about 6° C., between about 4° C. and about 5° C.

In another aspect, the invention provides a method of regenerating a cereal plant through tissue culture comprising the steps of (a) regenerating a plantlet from a callus, a somatic embryo or an immature embryo, and (b) temporarily immersing the plantlet in liquid medium at low temperature. In another embodiment the cereal plant is a wheat plant. Further embodiments provide that said plantlet is immersed for a period between 4 and 10 weeks and that the liquid medium comprises a carbon source, such as maltose. In another embodiment, said temperature is between 4° C. and 15° C., preferably 10° C.

Further provided is a method of increasing yield of a regenerated cereal plant through tissue culture comprising the steps of (a) regenerating a plantlet from a callus, a somatic embryo or an immature embryo, (b) temporarily immersing the plantlet in liquid medium at low temperature and (c) growing the plant. In another embodiment the cereal plant is a wheat plant. Further embodiments provide that said plantlet is immersed for a period between 4 and 10 weeks and that the liquid medium comprises a carbon source, such as maltose. In another embodiment, said temperature is between 4° C. and 15° C., preferably 10° C. In yet another embodiment, the increased yield is seed yield, with a yield increase of at least 2.1 fold compared to plants regenerated through tissue culture that were not temporarily immersed in liquid medium but were temporarily stored at low temperature. In another embodiment, the increased yield is due to an increase of the thousand seed weight, with a yield increase of at least 5% compared to plants regenerated through tissue culture that were not temporarily immersed in liquid medium but were temporarily stored at low temperature.

Yet another embodiment provides a method of producing a cereal plant through tissue culture comprising the steps of (a) regenerating a plantlet from a callus, a somatic embryo or an immature embryo, and (b) temporarily immersing the plantlet in liquid medium at low temperature. In another embodiment the cereal plant is a wheat plant. Further embodiments provide that said plantlet is immersed for a period between 4 and 10 weeks and that the liquid medium comprises a carbon source, such as maltose. In another embodiment, said temperature is between 4° C. and 15° C., preferably 10° C.

Also provided is a cereal plant obtained by the methods described above. In one embodiment, said cereal plant is a wheat plant. In yet another embodiment, said plant has an increased yield compared to a plant regenerated through tissue culture that were not temporarily immersed in liquid medium but were temporarily stored at low temperature. In one embodiment the increased yield is seed yield, with a yield increase of at least 2.1 fold compared to plants regenerated through tissue culture that were not temporarily immersed in liquid medium but were temporarily stored at low temperature. In another embodiment, the increased yield is due to an increase of the thousand seed weight, with a yield increase of at least 5% compared to plants regenerated through tissue culture that were not temporarily immersed in liquid medium but were temporarily stored at low temperature.

“Yield” as used herein can comprise yield of the plant or plant part which is harvested, such as seed, including seed oil content, seed protein content, seed weight (measured as thousand seed weight), seed number. When the yield is the seed yield, the yield increase achieved with the method described herein compared to plants regenerated through tissue culture that were not temporarily immersed in liquid medium but were temporarily stored at low temperature may be of at least about 2.1 fold, at least about 2.2 fold, at least about 2.3 fold or at least about 2.4 fold. When the yield is the seed weight, the yield increase achieved with the method described herein compared to plants regenerated through tissue culture that were not temporarily immersed in liquid medium but were temporarily stored at low temperature may be of at least about 5%, at least about 6%, at least about 7% or at least about 8%.

Cereal plants are well known in the art and are grasses cultivated for their edible grain. Examples of cereals are wheat, oats, rye, barley, rice, sorghum, and maize (corn).

The plants according to the invention may additionally contain an endogenous gene or a transgene, which confers herbicide resistance, such as the bar or pat gene, which confer resistance to glufosinate ammonium (Liberty®, Basta® or Ignite®) [EP 0 242 236 and EP 0 242 246 incorporated by reference]; or any modified EPSPS gene, such as the 2mEPSPS gene from maize [EP0 508 909 and EP 0 507 698 incorporated by reference], or glyphosate acetyltransferase, or glyphosate oxidoreductase, which confer resistance to glyphosate (RoundupReady®), or bromoxynitril nitrilase to confer bromoxynitril tolerance, or any modified AHAS gene, which confers tolerance to sulfonylureas, imidazolinones, sulfonylaminocarbonyltriazolinones, triazolopyrimidines or pyrimidyl(oxy/thio)benzoates, such as oilseed rape imidazolinone-tolerant mutants PM1 and PM2, currently marketed as Clearfield® canola. Further, the plants according to the invention may additionally contain an endogenous or a transgene which confers increased oil content or improved oil composition, such as a 12:0 ACP thioesteraseincrease to obtain high laureate, which confers pollination control, such as barnase under control of an anther-specific promoter to obtain male sterility, or barstar under control of an anther-specific promoter to confer restoration of male sterility, or such as the Ogura cytoplasmic male sterility and nuclear restorer of fertility.

The plants according to the invention may be further treated with a chemical compound, such as a chemical compound selected from the following lists: Herbicides: Clethodim, Clopyralid, Diclofop, Ethametsulfuron, Fluazifop, Glufosinate, Glyphosate, Metazachlor, Quinmerac, Quizalofop, Tepraloxydim, Trifluralin. Fungicides/PGRs: Azoxystrobin, N-[9-(dichloromethylene)-1,2,3,4-tetrahydro-1,4-methanonaphthalen-5-yl]-3-(difluoromethyl)-1-methyl-1H-pyrazole-4-carboxamide (Benzovindiflupyr, Benzodiflupyr), Bixafen, Boscalid, Carbendazim, Carboxin, Chlormequat-chloride, Coniothryrium minitans, Cyproconazole, Cyprodinil, Difenoconazole, Dimethomorph, Dimoxystrobin, Epoxiconazole, Famoxadone, Fluazinam, Fludioxonil, Fluopicolide, Fluopyram, Fluoxastrobin, Fluquinconazole, Flusilazole, Fluthianil, Flutriafol, Fluxapyroxad, Iprodione, Isopyrazam, Mefenoxam, Mepiquat-chloride, Metalaxyl, Metconazole, Metominostrobin, Paclobutrazole, Penflufen, Penthiopyrad, Picoxystrobin, Prochloraz, Prothioconazole, Pyraclostrobin, Sedaxane, Tebuconazole, Tetraconazole, Thiophanate-methyl, Thiram, Triadimenol, Trifloxystrobin, Bacillus firmus, Bacillus firmus strain 1-1582, Bacillus subtilis, Bacillus subtilis strain GB03, Bacillus subtilis strain QST 713, Bacillus pumulis, Bacillus. pumulis strain GB34. Insecticides: Acetamiprid, Aldicarb, Azadirachtin, Carbofuran, Chlorantraniliprole (Rynaxypyr), Clothianidin, Cyantraniliprole (Cyazypyr), (beta-)Cyfluthrin, gamma-Cyhalothrin, lambda-Cyhalothrin, Cypermethrin, Deltamethrin, Dimethoate, Dinetofuran, Ethiprole, Flonicamid, Flubendiamide, Fluensulfone, Fluopyram, Flupyradifurone, tau-Fluvalinate, Imicyafos, Imidacloprid, Metaflumizone, Methiocarb, Pymetrozine, Pyrifluquinazon, Spinetoram, Spinosad, Spirotetramate, Sulfoxaflor, Thiacloprid, Thiamethoxam, 1-(3-chloropyridin-2-yl)-N-[4-cyano-2-methyl-6-(methylcarbamoyl)phenyl]-3-{[5-(trifluoromethyl)-2H-tetrazol-2-yl]methyl}-1H-pyrazole-5-carboxamide, 1-(3-chloropyridin-2-yl)-N-[4-cyano-2-methyl-6-(methylcarbamoyl)phenyl]-3-{[5-(trifluoromethyl)-1H-tetrazol-1-yl]methyl}-1H-pyrazole-5-carboxamide, 1-{2-fluoro-4-methyl-5-[(2,2,2-trifluorethyl)sulfinyl]phenyl}-3-(trifluoromethyl)-1H-1,2,4-triazol-5-amine, (1E)-N-[(6-chloropyridin-3-yl)methyl]-N′-cyano-N-(2,2-difluoroethyl)ethanimidamide, Bacillus firmus, Bacillus firmus strain I-1582, Bacillus subtilis, Bacillus subtilis strain GB03, Bacillus subtilis strain QST 713, Metarhizium anisopliae F52.

### EXAMPLES

Unless stated otherwise in the Examples, all regeneration and plant tissue culture techniques are carried out according to the protocol as described in Yuji Ishida et al. 2015, Methods in Molecular Biology, 1223: 189-198.

### Example 1—Embryogenic Calli Low Temperature Storage

Embryogenic calli were obtained from wheat immature embryos as described in Yuji Ishida et al. 2015, Methods in Molecular Biology, 1223: 189-198, and cultivated on the following solid medium (for 1 L):

| | | |
|---|---|---|
| MS salts | 4.3 | g |
| LS vitamins (as described | 10 | ml |
| in Yuji Ishida et al, 2015, 100x) | | |
| Sucrose | 20 | g |
| MES monohydrate | 0.5 | g |
| CuSO4•5H2O (stock 5 mg/ml) | 500 | μl |
| Zeatin (stock 1 mg/ml) | 5 | ml |
| Gelrite | 3 | g |

| | |
|---|---|
| pH | 5.8 |

Embryogenic calli on this medium were placed for 1 to 3 weeks at 10° C., 16 h lightcycle (25-35 μmol m⁻²s⁻¹ before being put back at 22° C. (same medium, same light conditions) to enable further development of the shoots. No phenotypic deviations was observed between plants regenerated after this cold storage period and plants regenerated without this cold storage period.

### Example 2—Plantlets Low Temperature Storage

When shoots were well developed, plantlets were transferred to the following solid rooting medium (1 L):

| | | |
|---|---|---|
| L7 Macro salts (x10) | 100 | ml |
| L7 Micro salts (x1000) | 1 | ml |
| Fe EDTA (x200) | 5 | ml |
| Vitamins/Inositol (x200) | 5 | ml |
| Myo-inositol | 100 | mg |
| Maltose | 30 | g |
| Gelrite | 1.5 | g |

| | |
|---|---|
| pH | 5.7 |

Plantlets were grown at 22° C. for 2 to 3 weeks before being transferred to 4° C. for 4, 6 or 8 weeks. The phenotype of these plantlets was compared to the one of plantlets which were not exposed to cold and it was found that the cold treated plants were showing extensive browning of the leaves which was not observed in the not cold treated plants.

The plantlets were then transferred to soil and grown in a greenhouse until seed set. Flowering time, time to harvest, number of ears, number of seeds and thousand seed weight were scored. The results are shown in Table 1.

TABLE 1
average results, minimum and maximum
results obtained in brackets.

| | | | | | | Thousand |
|---|---|---|---|---|---|---|
| | Flowering | Time to | | Number | Number | seed |
| Treatment | time | harvest | | of ears | of seeds | weight |
| None | 41 days | 108 | days | 7 | 186 | ND |
| | | | | | (24-472) | |
| 4° C., | 32 days | 97 | days | 11 | 364 | 43.27 g |
| 4 weeks | | | | | (225-451) | |
| 4° C., | 33 days | 91 | days | 10 | 321 | 42.15 g |
| 6 weeks | | | | | (180-460) | |
| 4° C., | 35 days | 90 | days | 9 | 244 | 40.71 g |
| 8 weeks | | | | | (16-396) | |

ND: no data

Upon transfer to the greenhouse, the leaves of the cold treated plantlets were more yellow than the ones of the not cold treated plantlets, however, this color difference disappeared with time. The plants flowered on average a week earlier than those that were not cold treated and could be harvested 10 days to 3 weeks earlier. The cold treated plants produced more ears and those cold-treated for no more than 6 weeks had on average a higher yield with more seeds though without exceeding the maximum yield obtained for some of the untreated plants.

### Example 3—Temporary Immersion at Low Temperature

A surprising observation from the experiment described in Example 2 was that one leaf of a cold-treated plant which had grown into the solid rooting medium before the cold treatment had remained green throughout the cold storage period. To investigate this finding further, plantlets that had been transferred onto solid rooting medium as described in Example 2 were immerged in either water of liquid rooting medium (same recipe as in Example 2 but without Gelrite) before being transferred to 10° C. for 9 weeks. The phenotype of these plantlets was compared to the one of plantlets which were not exposed to cold.

The plantlets that had been immerged either in water or in liquid rooting medium did not show the yellowing of the leaves that was observed in the cold-treated plantlets of Example 2.

After transferring to soil, flowering time, time to harvest, number of ears, number of seeds and thousand seed weight were scored. The results are shown in Table 2.

TABLE 2
average results, minimum and maximum
results obtained in brackets

| | | | | | Thousand |
|---|---|---|---|---|---|
| | Flowering | Time to | Number | Number | seed |
| Treatment | time | harvest | of ears | of seeds | weight |
| 10° C., 9 | 42 days | 119 days | 10 | 245 | 39.43 g |
| weeks, no | | | (6-15) | (152-344) | |
| immersion | | | | | |
| 10° C., 9 | 35 days | 119 days | 20 | 491 | 41.38 g |
| weeks, water | | | (10-28) | (338-577) | |
| 10° C., 9 | 35 days | 119 days | 19 | 579 | 41.85 g |
| weeks, liquid | | | (17-21) | (510-666) | |
| rooting | | | | | |
| medium | | | | | |

The plants flowered on average a week earlier than those that were cold treated but not immersed but without affecting the harvest date. The immersed cold-treated plants produced on average twice more ears and had a higher yield with twice more seeds and bigger seeds (as indicated by the thousand seed weight results). In contrast to the seed increase observed when the cold-treatment is applied to non-immersed plantlets or to plantlets immersed in water, this seed yield increase has a strong penetrance since the lowest yielding plant that was immersed in liquid rooting media still has a seed yield of about 1.5 fold the one of the highest yielding plant which was not treated.

It was further observed that the plants which had been immersed in liquid rooting medium had a better ear quality compared to the non-immersed plants and the plants immersed in water during the cold-treatment.

The same experience was performed with a duration of 16 weeks at 10° C. All treated plants had a poor yield and quality indicating that a duration of 16 weeks is too long for this method.

### Example 4—Evaluation of Alternative Carbon Sources in the Rooting Medium

The superior results obtained when the plantlets were immersed in liquid rooting medium compared to water suggested a potential role for the carbon source present in the immersion solution.

To investigate this potential role, plantlets that had been transferred onto solid rooting medium as described in Example 2 were immerged in liquid rooting medium modified to replace the maltose with glucose, mannitol or sucrose before being transferred to 10° C. for 9 weeks.

The phenotype of these plantlets was compared to the one of plantlets which were immersed in liquid rooting medium as described in Example 3 before being transferred to 10° C. for 9 weeks.

1. A method of temporarily storing a cereal plant being regenerated through tissue culture comprising the steps of:
a. regenerating a plantlet from a callus, a somatic embryo or an immature embryo, and
b. temporarily immersing the plantlet in liquid medium at low temperature.

2. The method of claim 1, wherein the cereal plant is a wheat plant.

3. The method of claim 1, wherein the plantlet is immersed for a period between 4 and 10 weeks.

4. The method of claim 1, wherein the liquid medium comprises a carbon source.

5. The method of claim 1, wherein the temperature is between 4° C. and 15° C.

6. The method of claim 1, wherein the temperature is 10° C.

7. A method of regenerating a cereal plant through tissue culture comprising the steps of
a. regenerating a plantlet from a callus, a somatic embryo or an immature embryo, and
b. temporarily immersing the plantlet in liquid medium at low temperature.

8. The method of claim 7, wherein the cereal plant is a wheat plant.

9. The method of claim 7, wherein the plantlet is immersed for a period between 4 and 10 weeks.

10. The method of claim 7, wherein the liquid medium comprises a carbon source.

11. The method of claim 7, wherein the temperature is between 4° C. and 15° C.

12. The method of claim 7, wherein the temperature is 10° C.

13. A method of increasing yield of a cereal plant regenerated through tissue culture comprising the steps of:
a. regenerating a plantlet from a callus, a somatic embryo or an immature embryo,
b. temporarily immersing the plantlet in liquid medium at low temperature, and optionally
c. growing the plantlet.

14. The method of claim 13, wherein the cereal plant is a wheat plant.

15. The method of claim 13, wherein the plantlet is immersed for a period between 4 and 10 weeks.

16. The method of claim 13, wherein the liquid medium comprises a carbon source.

17. The method of claim 13, wherein the temperature is between 4° C. and 15° C.

18. The method of claim 13, wherein the temperature is 10° C.

19. The method of claim 13, wherein the increased yield is seed yield.

20. The method of claim 19, wherein the yield increase is of at least 2.1 fold compared to the yield of plants regenerated through tissue culture that were not temporarily immersed in liquid medium but were temporarily stored at low temperature.

21. The method of claim 13, wherein the increased yield is due to an increase of the thousand seed weight.

22. The method of claim 21, wherein the yield increase is of at least 5% compared to plants regenerated through tissue culture that were not temporarily immersed in liquid medium but temporarily stored at low temperature.

23. A method of producing a cereal plant through tissue culture comprising the steps of:
a. regenerating a plantlet from a callus, a somatic embryo or an immature embryo, and
b. temporarily immersing the plantlet in liquid medium at low temperature.

24. The method of claim 23, wherein the cereal plant is a wheat plant.

25. The method of claim 23, wherein the plantlet is immersed for a period between 4 and 10 weeks.

26. The method of claim 23, wherein the liquid medium comprises a carbon source.

27. The method of claim 23, wherein the temperature is between 4° C. and 15° C.

28. The method of claim 23, wherein the temperature is 10° C.

29. A cereal plant obtained by the method of claim 23.

30. The cereal plant of claim 29, which has an increased yield compared to a plant regenerated through tissue culture that were not temporarily immersed in liquid medium but were temporarily stored at low temperature.

31. The cereal plant of claim 29, wherein the increased yield is seed yield.

32. The cereal plant of claim 31, wherein the yield increase is of at least 2.1 fold.

33. The plant of claim 29, wherein the increased yield is due to an increase of the thousand seed weight.

34. The plant of claim 33, wherein the yield increase is of at least 5%.
